(19) United States
(12) Patent Application Publication
(10) Pub. No.: US 2018/0307964 A1
(43) Pub. Date: Oct. 25, 2018

(54) Abiotic Intelligent-Rendered Oracle

(71) Applicant: Anuthep Benja-Athon, New York, NY (US)

(72) Inventor: Anuthep Benja-Athon, New York, NY (US)

(73) Assignee: Anuthep Benja-Athon, (US)

(21) Appl. No.: 15/495,164

(22) Filed: Apr. 24, 2017

(51) Int. Cl. G06N 3/00; H04L 29/08; G06N 5/04

(52) U.S. Cl. CPC G06N 3/006; H04L 67/1097; G06Q 50/01; G06N 5/04; H04L 67/36

(57) ABSTRACT

The networked repositories for successively rendering comprehensive compilations of relations of subset*_n of relational data on continua of medicine, healthcare and related goods and ices as successive interactive universal network data-driven visualizations wherein successive interactive constituent visual-hierarchied flowing frameworks comprising individual and individual groups of said individual compilations of relations of subset*_n are successively rendered. The successive interactive constituent visual-hierarchied flowing frameworks for managing and guiding billions of lives and civilizations.

This is a continuation-in-part of application Ser. Nos. 14/992,773 entitled the Creation of Abiotic-Biotic Civilization filed Jan. 11, 2016, 14/606,956 entitled Billion Recounts of Events Ahead of Days filed Jan. 27, 2015 and 15/292,080 entitled EM Oracle filed Oct. 12, 2016.

### FIELD OF THE INVENTION

Abiotic Intelligent Rendered-Living Technology for trending lives.

### BACKGROUND OF THE INVENTION

The Abiotic Intelligent-Rendered Technology aka Abiotic Intelligent-Rendered Oracle henceforth AIR Oracle is the brain and heart of vitality of lives and civilizations. Despite decades of networking technologies like search engine, eCommerce and social media, billions of people's costs and standard of living, health, lives, longevity and future have neither been transformed nor improved to the earthshanking, revolutionary and evolutionary calibers.

AIR Oracle will continuously and perpetually networking billions of smartphones, other mobile devices and desktops, servers henceforth networked devices to universally 24/7 render, guide, trend, pattern, predict, extrapolate and transform people's lives and future and civilizations. Further to said EM Oracle, AIR Oracle is to 24/7 instantly and accurately guides, indicates, points to, procure and predict valuable and vital chains of goods and services vital to said billions to immensely and optimally improve their costs and standard of living, lives, longevity and future.

### SUMMARY OF THE INVENTION

In and among millions to billions of networked mobile communication devices for perpetually rendering successive interactive adaptable universal network data-driven visualizations of visual-hierarchied flowingframeworks (SIUNDV) wherein billions to infinite successive interactive adaptable visual-hierarchied constituent flowingframeworks (SIVSF) of and silhouetting said SIUNDV according to successive outcomes comprise correlations of relational subset*_n of fundamental medicine-health-healthcare and inherently and phenotypically related goods and services data being rendered by processor units and stored in and among said memories.

Successively rendered, adapted and expanded in and among touchscreens and viewing screens, said constituent SIVSF silhouetting SIUNDV for rendering, monitoring, trending, patterning, preventing, predicting, extrapolating and optimizing billions of people's standard of living, lives and future and civilizations.

### DIAGRAM

The Universe of Connected Constellations of Interactive Network Data Visualizations

### DESCRIPTION OF THE PREFERRED EMBODIMENTS

The present invention is a system of networked devices for managing, trending, patterning and guiding billions of lives and civilizations. A system of networked devices for diagnosing, surveilling, monitoring, trending, patterning, preventing, predicting, extrapolating, controlling and doctoring acute, subacute and chronic diseases, disorders, inflammations, traumas, sequelae and related predilections, trends, patterns, options, predictions and extrapolations of said renderings and procurements, prescriptions, utilizations, compliances and efficacies of inherently, phenotypically and consequentially related goods and services henceforth collectively known as Abiotic Intelligent-Rendered Oracle aka AIR Oracle by said networked devices.

The networking said networked devices comprising networked servers, smartphones, laptops and desktops having networked repositories comprising processor units and memories, touchscreens and viewing screens, cameras, microphones are readily achieved by the status quo Internet, commercial networking and security means and software including web browsers. Furthermore, more precise networking of said networked devices are achieved by the present invention, first, rendering AIR Oracle App to activate the innovative computer programs provided by the present invention to instruct said processor units to execute all functions described herein.

Second, said computer programs have the thesaurus software. Said thesaurus software networking and grouping said networked repositories according to correlations of of same, similar, linked, related and differential relational subset*_n of data in and among memories of said networked repositories, said thesaurus software for collaborating with said networked repositories to successively rendering, predicting, surveilling, monitoring, trending, patterning, preventing, predicting, extrapolating, controlling and doctoring said AIR Oracle and said compilations of relations and successive outcomes based on said thesaurus software successively rendering said compilations of relations and successive outcomes in professionals' and corresponding laypeople's languages in and among, first, said interactive templates and, second, said SIVSF successively silhouetting SIUNDV and, third, said SIUNDV.

said thesaurus software for collaborating with said networked repositories to render said networked repositories for successively rendering, predicting, surveilling, monitoring, trending, patterning, preventing, predicting, extrapolating, controlling and doctoring said AIR Oracle and said compilations of relations based on thesaurus software for successively rendering said compilations of relations and successive outcomes in professionals' and corresponding laypeople's languages in and among said SIVSF successively silhouetting SIUNDV and said SIUNDV.

Said professional and technical terminologies-lay terminologies thesaurus software for instructing said networked repositories to constitute and render relational professional and technical terminologies-lay terminologies analogies such as, but not limited to, English and non-English data in said SIVSF successively silhouetting SIUNDV in said touchscreens and viewing screens and in said memories. In other words, said thesaurus serves as the languages translator of data in and among said touchscreens and viewing screens wherein said data are displayed in languages, images and videos comprehensible and useable by and among laypeople and professional people.

Said innovative computer programs applies said thesaurus in and among networked servers and devices repositories.

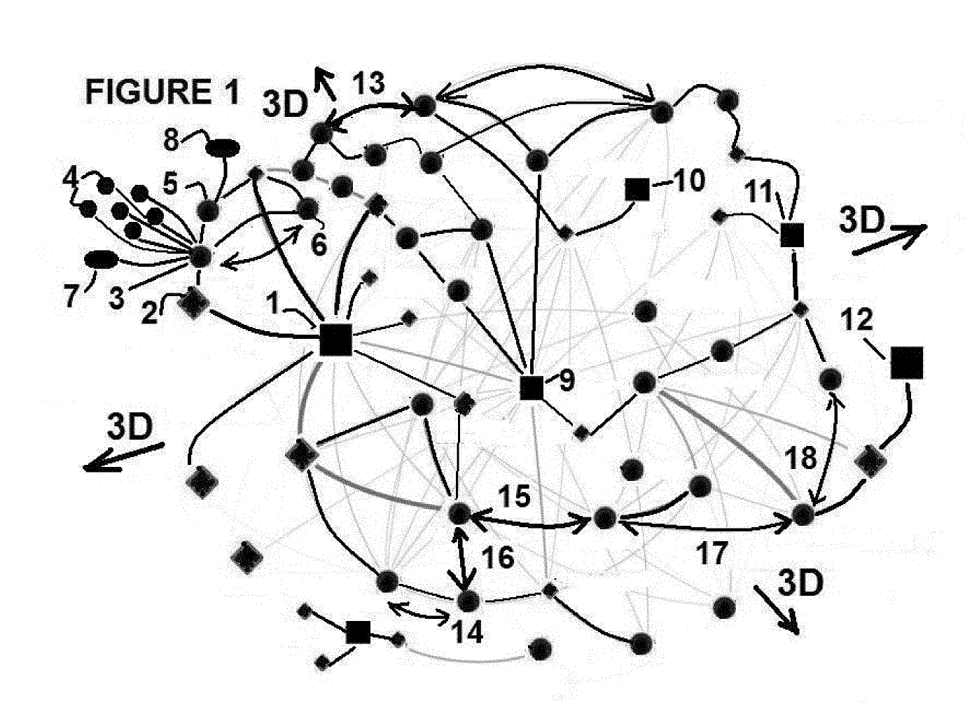

Brief Description of FIG. 1: The diagram representing is a schematics of one of numerous preferred embodiments of the present invention—described infra—wherein said constituent SIVSF successively silhouetting lighter SIUNDV associated with four 3D unidirectional arrows representing interactive adaptable and changeable, expandable and retractable multi-dimensional dynamic universe of constellations represented by squares 1, 9, 10, 11 and 12.

Said SIVSF including successive interactive dynamic adaptable and changeable projecting flowingframeworks building blocks for successively diagnosing, surveilling, monitoring, adapting, trending, patterning, preventing, protecting, predicting, controlling and doctoring said AIR Oracle in and among said SIVSF integral in and to said SIUNDV, consequently, said SIVSF including successive flowingframeworks building blocks for successively renderings, changes, adaptations, expansions of said predilections, trends, patterns, options, predictions and extrapolations in said AIR Oracle and in and among said compilations of relations according to said successive outcomes successively rendered by said processor units.

Said SIVSF including said projecting flowingframeworks building blocks comprising interactive adaptable, dynamic, changeable, expandable and retractable multi-dimensional interactive network data visualizations comprise interactive framework lines, nodes aka data points, spectrums, symbols, geometric configurations, tree traversals and graphs for diagnosing, surveilling, monitoring, adapting, trending, patterning, preventing, protecting, predicting, controlling and doctoring said AIR Oracle. Said projecting flowingframeworks building blocks for successively and progressively projecting said SIVSF in and among said SIUNDV in and among said constellations according to said successive outcomes rendered by and among said processor units.

The diagram—entitled The Universe of Connected Constellations of Interactive Network Data Visualizations represented by said lighter SIUNDV associated with four 3D unidirectional arrows in the realm of said networked devices is infinitely immense being continuously and infinitely rendered and changed—represented by—by said networked repositories in and among said millions to billions of touchscreens and viewing screens.

The diagram has several black square symbols henceforth squares to represent thousands (not shown) of squares 1, 9, 10, 11 and 12 representing connected constellation*_n comprising field*_n<industry*_n<enterprise*_n<specialty*_n<sector*_n<subsector*_n<subject*_n <topic*_n<heading*_n<category*_n<subset*_n of relational data henceforth known as medicine-health-healthcare relational subset*_n comprising correlations of linked, related and differential medicine-health-healthcare relational subset*_n comprising same, similar, linked, related and distinguishing relations of chronologically ordered relational sequences of timestamped data including images and videos inherently, phenotypically and consequentially related to relational subset*_n of goods*_n and service*_n data rendered by said processor units and stored in and among said memories, where symbols <, * and _n represent related to, diverse and plurality, respectively. Thus, among thousands of connected constellations, the universal fact is medicine-health-healthcare field or constellation—square 1—is the fundamental, core and existential constellation depended by said thousands of connecting constellations (not shown)—represented by squares 1, 9, 10, 11 and 12—embracing and pertaining to lives—people and animals—and civilizations.

Said relational subset*_n comprise fundamental data structure comprising fundamental medicine-health-healthcare subset*_n comprising medical and surgical specialty*_n-specific diagnosis*_n-specific plurality of medical-category*_n-specific chronologically ordered relational sequences of timestamped data including images and videos to which relate specialty*_n-specific linked diagnosis*_n-specific plurality of medical-category*-specific chronologically ordered relational sequences of timestamped data including images and videos to which relate specialty*_n-specific related diagnosis*_n-specific plurality of medical-category*_n-specific chronologically ordered timestamped data including images and video to which relate specialty*_n-specific differential diagnosis*_n-specific plurality of medical-category*_n-specific chronologically ordered relational sequences of timestamped data including images and videos and goods*_n and service*_n subset*_n—in said connecting constellations represented by squares 1, 9, 10, 11 and 12—inherently, phenotypically and consequentially related to said medicine-health-healthcare constellation.

Said successive outcomes comprise correlations of relational subset*_n of fundamental medicine-health-healthcare and inherently and phenotypically related goods and services data being rendered by processor units and stored in and among said memories.

Said constituent SIVSF silhouetting SIUNDV comprising individual compilations of relations and said comprehensive compilations of relations, respectively, according to said successive outcomes being rendered by said processor units.

Said constituent SIVSF silhouetting SIUNDV comprising said individual groups of said compilations of relations and said comprehensive compilations of relations, respectively, according to said successive outcomes being rendered by said processor units.

Well described in the Claims is one of the preferred embodiments of the renderings of said successive outcomes by, first, a plurality of electronic templates and, second, by interactive data-rendering and data-editing functions attaching to and associating with interactive framework lines, nodes aka data points, spectrums, symbols, geometric configurations, tree traversals and graphs in said SIVSF and SIUNDV wherein said interactive data-rendering and data-editing functions successively render, add, drag and drop, scroll, undo, cut, copy, paste, delete, find, find next, search, replace, go to, select said chronologically ordered relational sequences of timestamped data including images and videos and related subset*_n of data to successively rendering, adapting, editing and expanding said successive outcomes in and among said memories and, consequently, in and among said SIVSF and SIUNDV.

Said innovative computer programs instructing said processor units to scan, match and relate same, similar and distinguishing chronologically ordered relational sequences of timestamped data in and among said relational subset*_n of data in said individual compilations of relations and said individual groups of compilations of relations with said relational subset*_n of data in comprehensive compilations of relations to successively render said successive outcomes of said successive scans, matches and relations, said successive outcomes successively rendering, adapting, adding and editing said SIVSF integral in and to said constituent SIVSF silhouetting SIUNDV in and among said touchscreens and viewing screens and, consequently, said compilations of relations in and among said memories.

Based on said introduction, therefore, this Description describes a said constituent SIVSF silhouetting SIUNDV—representing billions to infinite constituent SIVSF silhouetting billions to infinite SIUNDV. Square 1 representing fundamental medicine-health-healthcare field or constellation. Connecting to square 1, 6 diamond symbols representing 85-100 diamonds (not shown) henceforth diamonds representing 85-100 medical, surgical and healthcare-specific specialties such as, but not limited to, allergy, anesthesiology, cardiology, dermatology, emergency medicine, neurology, pharmaceuticals and surgery, etc.

Connecting to diamond 2 is large circle 3 representing individual tens to hundreds of singular circles (not shown) attaching to a diamond, each circle representing a specialty*-specific diagnosis. Large circles 5 and 6—connecting to a diamond (not numbered)—representing yet another specialty*-specific diagnoses which representing yet other tens to hundreds of singular circles (not shown) attaching to said not-numbered diamond. Connecting to said large circle 3, a cluster of smaller circles 4 representing a specific diagnosis*-specific plurality of medical-category*_n-specific chronologically ordered relational sequences of timestamped data including images and videos. Said medical-category*_n comprise medical, health and healthcare categories of demographics, chief complaints, injuries, symptoms, signs, review of systems, physical examinations and findings, differential diagnoses, laboratory, test, pathology, diagnostics, measurements, pharmaceuticals, said physical interventions such as, but not limited to, surgeries, biological interventions such as, but not limited to, environmental changes and adaptations, genetic interventions such as, but not limited to, gene editings, individual idiosyncratic groups or bits of sequences of amino acids of chromosomes associating with said rendered heuristics and logics, etc. and chemical interventions such as, but not limited to, pharmaceuticals, chemotherapy, radiation therapy, therapeutics and engineering and related products, events and sequelae as consequences genetic data and engineering. For example, genetic and engineering of genetic materials in people and animals in the discoveries of diseases and disorders and their treatments such as, but not limited to, gene editings, etc.

Connecting to said large circle 3, a large circle 5 and a large circle 6—representing individual tens of singular circles attaching to individual large circles (not shown), so that any large circles connected to other large circles in and among a plurality of SIUNDV in and among constellations usually representing a diagnosis*—linked diagnosis*_n—related diagnosis*_n.

Connecting to said large circle 3 and large circle 5, ellipse 7 representing tens of differential diagnosis*_n related to said large circle 3 and ellipse 8 representing tens of differential diagnosis*_n related to said large circle 5.

Each bidirectional arrow among many bidirectional arrows 13, 14, 15, 16, 17 and 18—within or outside said flowingframeworks in the diagram—representing said interactive adaptable, dynamic, changeable, expandable and retractable multi-dimensional framework lines, nodes aka data points, spectrums, symbols, geometric configurations; tree traversals and graphs for successively and progressively projecting infinite SIVSF in and among said SIUNDV in and among said thousands of constellations—represented by squares 1, 9, 10, 11 and 12—according to said successive outcomes rendered by and among said processor units.

Therefore, based on said projecting and trending dynamic flowingframeworks of said constituent SIVSF in and among said SIUNDV in and among said constellations according to infinite numbers of successive outcomes rendered by and among said processor units, said infinite constituent SIVSF for successively and sequentially diagnosing, surveilling, monitoring, trending, patterning, preventing, predicting, extrapolating, controlling and doctoring acute, subacute and chronic diseases, disorders, inflammations, traumas, scquelae and related predilections, trends, patterns, options, predictions and extrapolations of said renderings and related procurements, prescriptions, utilizations, compliances and efficacies of related goods and services to significantly and effectively optimize people's standard of living, lives and future and civilizations.

Using the AIR Oracle App, through out the life of each and every person's compilations of relations of subset*_n and individual groups of people's compilations of relations of subset*_n successively and continously rendered, adapted, changed, edited and expanded as said infinite SIVSF in and among said SIUNDV to successively and continuously diagnose, surveil, monitor, adapt, trend, pattern, predict, extrapolate, control and doctor individual lives and individual groups of people's lives.

A case in point, based on said one representation, a group of several millions people—among thousands of individual groups of billions of people—will be or are afflicted by many common prevalent chronic diseases and disorders such as, but not limited to, diabetes mellitus, obesity, arthritis, aging or death, etc. As described supra, said people use networked devices to successively and continuously render infinite SIVSF in and among said SIUNDV to successively and continuously diagnose, surveil, monitor, adapt, trend, pattern, predict, extrapolate, control and doctor said many common prevalent chronic diseases and diagnoses of linked, related and differential diseases and disorders in the present and future. Consequently, for example, based on real-time renderings of said infinite SIVSF in and among said SIUNDV in and among said touchscreens and viewing screens, said groups of people can visualize and be informed of their status quo, trends, patterns, predictions and extrapolations of said chronic diseases and disorders and related subsets_n of data of inherently, phenotypically and consequentially relational goods*_n and service*_n data optimally and cost-effectively available to them. Therefore, they will be able to surveil, monitor, adapt, avoid and prepare for said present and future diseases and disorders and related afflictions and complications. Thus, said infinite SIVSF in and among said SIUNDV in and among said touchscreens and viewing screens optimizing costs and standard of living, lives and future for billions of people and civilizations.

Although said black-colored silhouetted flowingframeworks—in diagram [and FIG. 2]—to represent myriads of interactive multi-dimensional adaptable, changeable, expandable and retractable flowingframeworks, it will be appreciated by and obvious to those skilled in the art and science that said interactive network data visualizations will have and use a myriad of combinations of visual hierarchies for achieving the goals and objectives of the present invention as described infra. Said visual hierarchies are such as, but not limited to, hierarchies of diverse and spectrums of colors, colored and varying dimensions of symbols, signs, said chronologically ordered relational sequences of timestamped data in and among said compilations of relations, accentuated texts and numbers, colored, calibers, dimensions and configurations of connecting lines and the likes, configurations, figures, images and video, etc. and any combination thereof are and will be used to said multi-dimensional dynamic interactive network visual-hierarchied data visualizations. Said visual hierarchies are used for the entire multi-dimensional dynamic interactive network visual-hierarchied data visualizations of flowingframeworks and flowingdata of connected visual-hierarchied nodes and interactive visual-hierarchied data in said interactive data-rendering and data-editing functions. Thus, in said constellations, said black silhouetted flowingframeworks and lighter constellations flowingframeworks are rendered and successively rendered as myriads of said interactive network visual-hierarchied data visualizations of visual-hierarchied flowingframeworks and flowingData and adaptations, variations and derivatives will be made and used to achieve the objectives of the invention without departing from the spirit and scope of the specification of the present invention. Thus, said networked repositories rendering and successively rendering said infinite quantities of interactive adaptable, changeable, expandable and retractable dynamic multi-dimensional visual-hierarchied flowingframeworks of interactive network visual-hierarchied data visualizations of of flowingdata of connected visual-hierarchied nodes integral in and to said infinitely large interactive adaptable, expandable and retractable multi-dimensional dynamic interactive network data visualizations of constellations visual-hierarchied flowingframeworks of the Constellation in and among millions to billions touchscreens and viewing screens of said networked devices.

Although several interactive network visual-hierarchied data visualizations of visual-hierarchied flowingData—schematically represented in said diagram and described herein—representing one of the preferred embodiments of the present invention, it will be appreciated by and obvious to those skilled in the art and science that a myriad of interactive network visualizations of visual-hierarchied flowingData such as, but not limited to, various interactive spectrums and gradients of connected visual-hierarchied data points, interactive graphs and interactive network visualizations of flowingdata of connected visual-hierarchied data points and adaptations, variations and derivatives will be made and used to achieve the objectives of the invention without departing from the spirit and scope of the specification of the present invention.

Although various preferred embodiments of the present invention have been described, it will be appreciated by and obvious to those skilled in the art that adaptations, variations and derivatives of said embodiments are and will be made and achieved for people, animals and machines and objects without departing from the spirit and scope of the specification of the present invention.

1. A system of networked repositories for diagnosing, surveilling, monitoring, trending, patterning, preventing, predicting, extrapolating, controlling and doctoring acute, subacute and chronic diseases, disorders, inflammations, traumas, sequelae and related predilections, trends, patterns, options, predictions and extrapolations of said renderings and related procurements, prescriptions, utilizations, compliances and efficacies of related goods and se ices people's lives henceforth collectively known as Abiotic Intelligent-Rendered Oracle also known as AIR Oracle, said networked repositories for successively render g adaptable, changeable, expandable and retractable dynamic multi-dimensional interactive universal network data-driven visualizations comprising interactive universal network data-driven flowingframeworks of visual-hierarchied flowingdata of connected visual-hierarched data points aka visual-hierachied nodes henceforth collectively known as SIUNDV in and among connected constellations wherein infinite individual connected assortments of interactive adaptable, changeable, expandable and retractable multi-dimensional dynamic network data-driven visualizations of visual-hierarchied flowingframeworks of visual-hierarchied flowingdata of connected interactive visual-hierarchied nodes henceforth known as successive integral visual-hierarchied silhouetted flowingframeworks having interactive dynamic adaptable and changeable projecting flowingframeworks building blocks collectively known as SIVSF integral in and to said SIUNDV in and along said connected constellations in and among touchscreens and viewing screens are successively rendered, adapted and expanded according to successive outcomes in said SIVSF silhouetting said SIUNDV in and among said touchscreens, viewing screens and memories rendered by processor units of networked devices wherein a plurality of interactive data-rendering and data-editing functions attaching to and associating with said SIVSF and SIUNDV, said data-rendering and data-editing functions for successively diagnosing, surveilling, monitoring, trending, patterning, preventing, predicting, extrapolating, controlling and doctoring said AIR Oracle, said data-rendering and data-editing functions for successively rendering, adapting, changing, expanding and retracting said compilations of relations in and among said and successive SIVSF and UINDV in said constellations and memories, said data-rendering and data-editing functions for cybersecurely rendering, expanding and editing said compilations of relations in and among said flowingframeworks and said memories, said data-rendering and data-editing functions for defining cybersecured definitions, characteristics and limitations of data being rendered and permitted into said memories to prevent perpetrations of malfeasances on and protect said system, said flowingframeworks comprising visual hierarchies for successively rendering, predicting, surveilling, monitoring, trending, patterning, preventing, predicting, extrapolating, controlling and doctoring said AIR Oracle and said compilations of relations, said system comprises:
networks of said networked devices comprising worked servers, smartphones, laptops and desktops having said networked repositories, said touchscreens and viewing screens, cameras, microphones, said networked repositories comprising said processor units for rendering said networks based on said compilations of relations, said processor units for processing, rendering and expanding said individual and individual groups of compilations of relations with said comprehensive compilations of relations, the innovative computer programs attaching to said processor units attached to said memories, said innovative computer programs attaching aid processor units, said innovative computer programs for instructing said processor units to successively render and expand said compilations of relations in said connected constellations. and in and among said memories, said memories for storing said individual and individual groups of compilations of relations with said comprehensive compilations of relations in said successive SIVSF*_n and said IUDDV*_n where * and _n represent diverse and plurality, respectively, and memories, software including said innovative computer programs, commercial operating systems for managing said networked servers and devices, the Internet, commercial networking means, security means and software including web browsers;
said innovative computer programs have the thesaurus software for networking and grouping said networked repositories according to correlations of of same similar, linked, related and differential relational subset*_n of data in and among memories of said networked repositories, said thesaurus software for collaborating with said networked repositories to successively rendering, predicting, surveilling, monitoring, trending, patterning, preventing, predicting, extrapolating, controlling and doctoring said AIR Oracle and said compilations of relations and successive outcomes based on said thesaurus software successively rendering said compilations of relations and successive outcomes in professionals' and corresponding laypeople's languages in and among, first, said interactive templates and, second, said SIVSF successively silhouetting SIUNDV and, third, said SIUNDV;
said thesaurus software for collaborating with said networked repositories to render said networked repositories for successively rendering, predicting, surveilling, monitoring, trending, patterning, preventing, predicting, extrapolating, controlling and doctoring said AIR Oracle and said compilations of relations based on thesaurus software for successively rendering said compilations of relations and successive outcomes in professionals' and corresponding laypeople's languages in and among said SIVSF successively silhouetting SIUNDV and said SIUNDV;
said innovative programs for instructing said processor units to successively render:
AIR Oracle App for activating innovative computer programs said processor units for executing all functions described herein;
said processor units for continuously and infinitely rendering, adapting and changing said correlations of relations in successively rendered, adapted, changed, edited and expanded interactive integral visual-hierarchied silhouetted flowingframeworks successively silhouetting said interactive universal network data-driven flowingframeworks wherein said successive SIVSF for successively diagnosing, surveilling, monitoring, adapting, trending, patterning, preventing, predicting and controlling and doctoring said AIR Oracle in and among said SIUNDV and said compilations of relations in and among memories according to said successive outcomes;
said successive outcomes for rendering said AIR Oracle in and among said SIVSF successively silhouetting SIUNDV and said SIUNDV in and among memories according to said successive outcomes;
said processor units for successively rendering, adapting, changing and editing said SIVSF for successively silhouetting said SIUNDV wherein SIVSF for successively diagnosing, surveilling, monitoring, adapting, trending, patterning, preventing, predicting and controlling and doctor said AIR Oracle in and among said interactive universal network data-driven flowingframeworks according to said successive outomes being rendered and successively rendered by said processor units;
said SIVSF including successive interactive dynamic adaptable and changeable projecting flowingframeworks building blocks for successively diagnosing, surveilling, monitoring, adapting, trending, patterning, preventing, protecting, predicting, controlling and doctoring said AIR Oracle in and among said SIVSF integral in and to said SIUNDv, consequently, said SIVSF including successive flowingframeworks building blocks for successively renderings, changes, adaptations, expansions of said predilections, trends, patterns, options, predictions and extrapolations in said AIR Oracle and in and among said compilations of relations according said successive outcomes successively rendered by said processor units;
said SIVSF including said projecting flowingframeworks building blocks comprising interactive adaptable dynamic, changeable, expandable and retractable multi-dimensional interactive network data visualizations comprise interactive framework lines, nodes aka data points, spectrums, symbols, geometric configurations, tree traversals and graphs for diagnosing, surveilling, monitoring, adapting, trending, patterning, preventing, protecting, predicting, controlling and doctoring said AIR Oracle;
said projecting flowingframeworks building blocks for successively and progressively projecting said SIVSF in and among said SIUNDV in and among said constellations according to said successive outcomes rendered by and among said processor units;
first a plurality of interactive electronic templates for rendering and successively rendering said successive outcomes based on said correlations of individual compilations of relations and individual groups of said indivdual compilations of relations with comprehensive compilations of relations based on correlations of linked, related and differential medicine-health healthcare relational subset*_n comprising same, linked, related and distinguishing data on chronologically ordered relational sequences of timestamped data including images and videos and relational subset*_n of goods*_n and service*_n data inherently, phenotypically and consequentially related to said medicine-health-healthcare relational subset*_n rendered by said processor units and stored in and among said memories, where symbols<, * and _n represent related to, diverse and plurality, respectively;
second, said interactive data-rendering and data-editing functions attaching to and associating with said SIVSF and SIUNDV, said interactive data-rendering and data-editing functions for successively render, add, drag and drop, scroll, undo, cut, copy, paste, delete, find, find next. search, replace, go to, select said chronologically ordered relational sequences of timestamped data including images and videos and related subset*_n of data in and among said interactive framework lines, nodes aka data points, spectrums, symbols, geometric configurations, tree traversals and graphs, said data-rendering and data-editing for successively rendering, adapting, editing and expanding said successive outcomes in and among said memories and, consequently, in and among said SIVSF and SIUNDV.
Said constituent SIVSF silhouetting SIUNDV comprising said individual compilations of relations and said comprehensive compilations of relations, respectively, according to said successive outcomes being rendered by said processor units;
Said constituent SIVSF silhouetting SIUNDV comprising said individual groups of said compilations of relations and said SIUNDV, respectively, according to said successive outcomes being rendered by said processor units;
said SIVSF integral in and to said SIUNDV in and among touchscreens and viewing screens whenever successively rendered, adapted and edited individual compilations of relations and successively rendered, adapted and edited individual groups of said individual compilations of relations successively correlated with said comprehensive compilations of relations by said processor units;
said interactive dynamic adaptable and changeable projecting flowingframeworks building blocks for diagnosing, surveilling, monitoring, trending, patterning, preventing, predicting, extrapolating, controlling and doctoring said AIR Oracle according to said successive outcomes being rendered and successively rendered by said processor units;
said interactive data-rendering and data-editing functions for diagnosing, surveilling, monitoring, trending, patterning, preventing, predicting, extrapolating, controlling and doctoring said AIR Oracle:
said visual hierarchies for successively and sequentially diagnosing, surveilling, monitoring, trending, patterning, preventing, predicting, extrapolating, controlling and doctoring said AIR Oracle in and among said SIVSF successively silhouetting SIUNDV and in said successive outcomes in and among memories; and
said innovative computer programs for instructing said processor units for successively scanning, matching and relating same, similar and distinguishing chronologically ordered relational sequences of timestamped data in and among said relational subset of data in said individual compilations of relations and said individual groups of compilations of relations with said relational subset*_n of data in comprehensive compilations of relations to successively render said successive outcomes of sad successive scans, matches and relations, said successive outcomes for successively rendering, adapting, adding and editing said SIVSF especially said interactive dynamic adaptable and changeable projecting flowingframeworks building blocks integral in and to said SIUNDV and said SIUNDV in and among said touchscreens and viewing screens and, consequently, said compilations of relations in and among said memories.

2. The system according to claim 1 wherein said SIVSF integral in and to said SIUNDV and said SIUNDV comprise said interactive, adaptable, changeable, expandable and retractable framework lines, nodes aka data points, spectrums, symbols, geometric configurations, tree traversals and graphs for diagnosing, surveilling, monitoring, adapting, trending, patterning. preventing, protecting, predicting, controlling and doctoring said AIR Oracle; and
said interactive framework lines, nodes aka data points, spectrums, symbols, geometric configurations, tree traversals and graphs for comprising, expressing and displaying said chronologically ordered relational sequences of timestamped data hiding images and videos and related subset*_n of data of Inherently, phenotypically and consequentially relational goods*_n and service*_n data specifically relevant to framework lines, nodes aka data points, spectrums, symbols, geometric configurations, tree traversals and graphs.

3. The system according to claim 1 wherein said interactive dynamic adaptable and changeable projecting flowingframeworks building blocks in SIVSF integral in and to said SIUNDV, said projecting flowingframeworks building blocks for diagnosing, surveilling, monitoring, adapting, trending, patterning, preventing, protecting, predicting, controlling and doctoring said AIR Oracle.

4. The system according to claim 1 wherein said successive outcomes comprise said fundamental, core or existential medicine-health-healthcare relational subset*_n comprising correlations of linked, related and differential medicine-health-healthcare relational subset*_n comprising same, similar, linked, related and distinguishing relations of chronologically ordered relational sequences of timestamped data including images and videos inherently, phenotypically and consequentially related to relational subset*_n of goods*_n and service*_n data rendered by said processor units and stored in and among said memories.

5. The system according to claim 1 wherein said successive outcomes comprise successive correlations of individual compilations of relations of subset*_n with comprehensive compilations of relations of subset*_n in individual electronic templates;
individual groups of said compilations of relations of subset*_n with comprehensive compilations of relations of subset*_n in and among said electronic templates;
individual groups of said compilations of relations of subset*_n with comprehensive compilations of relations of subset*_n by said interactive data-rendering and data-editing functions add, drag and drop, scroll, undo, cut, copy, paste, delete, find, find next, search, replace, go to, select data in said subset*_n of chronologically ordered relational sequences of timestamped data including images and videos and related goods*_n and service*_n data being expressed and displayed in said interactive framework lines, nodes aka data points, spectrums, symbols, geometric configurations, tree traversals and graphs in and among said SIVSF and SIUNDV.

6. The relational subset according to claim 1 comprise fundamental data structure comprising fundamental medicine-health-healthcare subset*_n comprising medical and surgical specialty*_n-specific diagnosis*_n-specific plurality of medical-category*_n-specific chronologically ordered relational sequences of timestamped data including images and videos to which relate specialty*_n-specific linked diagnosis*_n-specific plurality of medical-category*-specific chronologically ordered relational sequences of timestamped data including images and videos to which relate specialty*_n-specific related diagnosis*_{——}n-specific plurality of medical-category*_n-specific chronologically ordered timestamped data including images and video to which relate specialty*_n-specific differential diagnosis*_n-specific plurality of medical-category*_n-specific chronologically ordered relational sequences of timestamped data including images and videos and goods*_n and service*_n subset*_n. inherently, phenotypically and consequentially related to said medicine-health-healthcare subset*_n;
data on said networked devices identification*_n, where symbols * and _n mean diverse and plurality, respectively.

7. The goods*_n and service*_n subset*_n according to claim 7 comprise fundamental data structure comprising relational goods*_n and service*_n data>heading*_n>category*_n>topic*_n>subject*_n>subject*_n>subject*_n>subsector*_n>sector*_n>specialty*_n>>fold*_n of business*_n and industry*_n relating to said subset*_n of people's health, healthcare, diseases, disorders and longevity, said relational fgoods*_n and service*_n data inherently, phenotypically and consequentially related to said relations of chronologically ordered relational sequences of timestamped data including images and videos, where symbols > and * and _n lean relating to, diverse and plurality, respectively,

8. The system according to claim 1 wherein said plurality of interactive chronic templates for rendering and successively rendering said compilation of relations in and among said networked repositories, said electronic templates for rendering and successively rendering
individual compilations of relations based on correlations of linked, related and differential medicine-health-healthcare relational subset*_n comprising same, similar, linked, related and distinguishing data on chronologically ordered relational sequences of timestamped data including images and videos and relational subset*_n of goods*_n and service*_n data inherently, phenotypically and consequentially related to said medicine-health-healthcare relational subset*_n rendered by said processor units and stored in individual memories;
individual groups of said individual compilations of relations in and among said memories based on correlations of linked, related and differential medicine-health-healthcare relational subset*_n having same, similar, linked, related and distinguishing fundamental medicine-health-healthcare subset*_n rendered by said processor units and stored in and among said memories;
comprehensive compilations of said individual groups of said individual compilations of relations rendered by said processor units and stored in and among said memories; and said successive outcomes.

9. The system according to claim 1 wherein said interactive data-rendering and data-editing functions for successively rendering, surveilling, monitoring, adapting, trending, patterning, predicting, extrapolating, controlling and doctoring said AIR Oracle in said SIVSF integral in and to said SIUNDV and compilations of relations in and among said memories comprise said interactive data-rendering and data-editing functions attaching to and associating with interactive framework lines, nodes aka data points, spectrums, symbols, geometric configurations, tree traversals and graphs in and among said SIVSF and SIUNDV:
said interactive framework lines, nodes aka data points, spectrums, symbols, geometric configurations, tree traversals and graphs for interactively comprising, expressing and displaying chronologically ordered relational sequences of timestamped data including images and videos and related subset*_n of data of inherently, phenotypically and consequentially relational goods*_n and service*_n data specifically relevant to each node in said trees wherein data are rendered interactive by said processor units: and
said interactive data-rendering and data-editing functions for successively render, add, drag and drop, scroll, undo, cut, copy, paste, delete, find, find next, search, replace, go to, select said chronologically ordered relational sequences of timestamped data including images and videos and related subset*_n of data in and among said interactive framework lines, nodes aka data points, spectrums, symbols, geometric configurations, tree traversals and graphs, said, data-rendering and data-editing functions for successively rendering, adapting, editing and. expanding said successive outcomes in and among said SIVSF and SIUNDV and said memories.

10. The system according to claim 1 wherein said data-rendering and data-editing functions for editing, cutting, pasting, adding, and deleting relational sequences of data in, link to and relate with data of in and among said interactive network visualizations of adaptable, changeable, expandable and retractable visual-hierarchied flowingdata of adaptable connected visual-hierarchied nodes and said compilations in and among said memories of networked repositories.

11. The system according to claim 1 wherein said interactive data-rendering and data-editing functions for diagnosing, surveilling, monitoring, trending, patterning, preventing, predicting. extrapolating, controlling and doctoring said AIR Oracle comprising said data-rendering and data-editing functions for successively rendering, changing, adapting, expanding and retracting said SIVSF integral in and to said SIUNDV wherein said successive adaptable, changeable, expandable and retractable visual-hierarchied framework lines, nodes, data points, spectrums, symbols, geometric configurations, tree traversals and graphs, etc., and any combinations thereof successively adapting, changing, expanding and retracting in said SIVSF successively silhouetting said SIUNDV in and among said connected constellations in and among touchscreens and viewing screens according to said successive compilations of relations in and among said memories rendered by said processor units; and
in said SIVSF, said successive interactive flowingframeworks building blocks for diagnosing. surveilling, monitoring, adapting, trending, patterning, preventing, protecting, predicting, controlling and doctoring said AIR Oracle and said successive compilations of relations in and among said memories.

12. The system according to claim 1 wherein said cybersecured definitions characteristics and limitations of data comprise said processor units-rendered subset*_n of data and relations among said data in related subset*_n for defining, characterizing and limiting said input data into said data-rendering and data-editing functions.

13. The system according to claim 1 wherein said visual hierarchies for sequentially rendering, adapting, expanding and retracting successive, predilections, trends, patterns, options, predictions and extrapolations comprise interactive frameworks building blocks comprising said said interactive flowingframeworks building blocks for diagnosing, surveilling, monitoring, adapting, trending, patterning, preventing, protecting, predicting, controlling and doctoring said AIR Oracle; and
said interactive data-rendering and data-editing functions for diagnosing, surveilling, monitoring, trending, patterning, preventing, predicting, extrapolating, controlling and doctoring AIR Oracle.

14. In the system according to claim 1, a method of said networked repositories successively rendering adaptable, changeable, expandable and retractable dynamic multi-dimensional interactive universal network data-driven visualizations comprising infinite interactive universal network data-driven flowingframeworks of visual-hierarchied flowingdata of connected visual-hierarchied data points aka visual-hierarchied nodes collectively known as SIUNDV in and among connected constellations wherein infinite individual connected assortments of interactive adaptable, changeable, expandable and retractable multi-dimensional dynamic network data-driven visualizations of visual-hierarchied flowingframeworks of visual-hierarchied flowingdata of connected interactive visual-hierarchied nodes having interactive dynamic adaptable and changeable projecting flowingframeworks building blocks henceforth collectively known as successive integral visual-hierarchied silhouetted flowingframeworks aka SIVSF integral in and to said SIUNDV in and among said connected constellations in and among touchscreens and viewing screens are successively rendered, adapted and, expanded according to successive outcomes of processor units correlating and relating individual compilations of relations of subset*_n of data comprising successive correlations of relations of successively rendered, correlated and related subset*_n of data by said processor units henceforth compilations of relations, individual groups of said compilations of relations with comprehensive compilations of relations first in and among said networked repositories and, second, in and among said SIVSF silhouetting said SIUNDV in and among said touchscreens, viewing screens and memories, a plurality of interactive data-rendering and data-editing functions attaching to and associating with SIVSF, and in and among said SIVSF silhouetting said SIUNDV, a plurality of data-rendering and data-editing functions successively diagnosing, surveilling, monitoring, trending, patterning, preventing, predicting, extrapolating, controlling and doctoring said AIR Oracle, said data-rendering and data-editing functions successively rendering, adapting, changing, expanding and retracting said compilations of relations in and among said and successive SIVSF and SIUNDV in said constellations and memories, said data-rendering and data-editing functions cybersecurely rendering, expanding and editing said compilations of relations in and among said flowingframeworks and said memories, said data-rendering and data-editing functions defining cybersecured definitions, characteristics and limitations of data being rendered and permitted into said memories to prevent perpetrations of malfeasances on and protect said system, said flowingframeworks comprising visual hierarchies successively rendering, predicting, surveilling, monitoring, trending, patterning, preventing, predicting, extrapolating, controlling and doctoring said AIR Oracle and said compilations of relations, said method comprises processes of:
networking said networked devices comprising networked servers, smartphones, laptops and desktops having said networked repositories, said touchscreens and viewing screens, cameras, microphones, said networked repositories comprising said processor units rendering said networks based on said compilations of relations, said processor units processing, rendering and expanding said individual and individual groups of compilations of relations with said comprehensive compilations of relations, the innovative computer programs attaching to said processor units attached to said memories, said innovative computer programs attaching to said processor units, said innovative computer programs instructing said processor units to successively render and expand said compilations of relations in said connected constellations and in and among said memories, said memories storing said individual and individual groups of compilations of relations with said comprehensive compilations of relations in said successive SIVSF*_n and said IUDDV*_n where * and _n represent diverse and plurality, respectively, and memories, software including said innovative computer programs, commercial operating systems managing said networked servers and devices, the Internet, commercial networking means, security means and software including web browsers.
said innovative computer programs instructing said processor units to successively and continuously:
rendering AIR Oracle App activating innovative computer programs said processor units executing all functions described herein;
rendering the thesaurus software, said thesaurus software networking and grouping said networked repositories according to correlations of of same, similar, linked, related and differential relational subset*_n of data in and among memories of said networked repositories, said thesaurus software collaborating with said networked repositories to successively rendering, predicting, surveilling, monitoring, trending, patterning, preventing, predicting, extrapolating, controlling and doctoring said AIR Oracle and said compilations of relations based on said thesaurus software successively rendering said compilations of relations in professionals' and corresponding laypeople's languages in and among said SIVSF successively silhouetting SIUNDV and said SIUNDV;
rendering a plurality of interactive electronic templates for rendering and successively rendering said successive outcomes based on said correlations of individual compilations of relations and individual groups of said individual compilations of relations with comprehensive compilations of relations based on correlations of linked, related and differential medicine-health-healthcare relational subset*_n comprising same, similar, linked, related and distinguishing data on chronologically ordered relational sequences of timestamped data including images and videos and relational subset*_n of goods*_n and service*_n data inherently, phenotypically and consequentially related to said medicine-health-healthcare relational subset*_n rendered by said processor units and stored in and among said memories, where symbols<, * and _n represent related to, diverse and plurality, respectively;
rendering said interactive data-rendering and data-editing functions attaching to and associating with said SIVSF and SIUNDV, said interactive data-rendering and data-editing functions for successively render, add, drag and drop, scroll, undo cut, copy, paste, delete, find, find next, search, replace, go to, select said chronologically ordered relational sequences of timestamped data including images and videos and related subset*_n of data in and among said interactive framework lines, nodes aka data points, spectrums, symbols, geometric configurations, tree traversals and graphs, said data-rendering and data-editing functions for successively rendering, adapting, editing and expanding said successive outcomes in and strong said memories and, consequently, in and among said SIVSF and SIUNDV;
scanning, match ng and relating same, similar and distinguishing chronologically ordered relational sequences of timestamped data in and among said relational subset*_n of data in said individual compilations of relations and said individual groups of compilations of relations with said relational subset*_n of data in comprehensive compilations of relations to successively render said successive outcomes of said successive scans, matches and relations said successive outcomes successively rendering, adapting, adding and editing said SIVSF integral in and to said SIUNDV and said SIUNDV in and among said touchscreens and viewing screens and, consequently, said compilations of relations in and among said memories;
rendering said constituent SIVSF silhouetting SIUNDV comprising said individual compilations of relations and said comprehensive compilations of relations, respectively, according to said successive outcomes being rendered by said processor units;
rendering said constituent SIVSF silhouetting SIUNDV comprising said individual groups of said compilations of relations and and said comprehensive compilations of relations, respectively, according to said successive outcomes being rendered by said processor units;
rendering said interactive dynamic adaptable and changeable projecting flowingframeworks building blocks to projecting said SIVSF to successively diagnosing, surveilling, monitoring, trending, patterning, preventing, predicting, extrapolating, controlling and doctoring said AIR Oracle in said SIVSF integral in and to SIUNDV and successively and progressively projecting said SIVSF to silhouet said SIUNDV according to said successive outcomes rendered by and among said processor units;
rendering, adapting, changing and expanding said successive outcomes in and among said memories and, consequently, in SIVSF integral in and to said SIUNDV and SIVSF successive silhouetting said SIUNDV wherein said successive outcomes are being continuously and perpetually rendered, adapted, changed and expanded by functions causing said SIVSF to successively diagnosing, surveilling, monitoring, adapting, trending, patterning, preventing, protecting, predicting, controlling and doctoring said AIR Oracle by said interactive framework lines, nodes aka data points, spectrums, symbols, geometric configurations, tree traversals and graphs interactively comprising, expressing and displaying chronologically ordered relational sequences of tirnestamped data including images and videos and related subset*_n of data of inherently, phenotypically and consequentially relational goods*_n and service*_n data specifically relevant to interactive framework lines, nodes aka data points, spectrums, symbols, geometric configurations, tree traversals and graphs wherein data are rendered interactive by said processor units; and
attaching and associating said interactive data-rendering and, data-editing functions with said interactive framework lines, nodes aka data points, spectrums, symbols, geometric configurations, tree traversals and graphs wherein said interactive data-rendering and data-editing functions successively rendering, surveilling, monitoring, adapting, trending, patterning, predicting, extrapolating, controlling and doctoring said AIR Oracle by successively render, add, drag and drop, scroll, undo, cut, copy, paste, delete, find, find next, search, replace, go to, select said chronologically ordered relational sequences of timestamped data including images and videos and related subset*_n of data among said interactive framework lines, nodes aka data points, spectrums, symbols, geometric configurations, tree traversals and graphs;
assigning said data-rendering and data-editing functions to cybersecurely rendering, expanding and editing said compilations of relations in and among said SIVSF integral in and to said SIUNDV and said memories by said data-rendering and data-editing functions defining cybersecured definitions, characteristics and limitations of data being rendered and permitted it to said memories to prevent perpetrations of malfeasances on and protect said system; and
rendering visual hierarchies in said SIVSF integral in and to said SIUNDV and SIUNDV to successively rendering, predicting, surveilling, monitoring, trending, patterning, preventing, predicting, extrapolating, controlling and doctoring said AIR Oracle and said compilations of relations in and among said memories.

15. The method according to claim 14 wherein said process of said innovative computer programs instructing said processor units to successively and continuously scanning, matching and relating same, similar and distinguishing chronologically ordered relational sequences of timestamped data and among said relational subset*_n of data in said individual compilations of relations and said individual groups of compilations of relations with said relational subset*_n of data in comprehensive compilations of relations to successively render said successive outcomes of said successive scans, matches and relations, said process comprises processes of
first, a plurality of interactive electronic templates for rendering and successively rendering said successive outcomes comprising correlations of compilations of relations of linked, related and differential data comprising same, similar, linked, related and distinguishing relations of chronologically ordered relational sequences of timestamped data including images and videos and related subset*_n of data of inherently, phenotypically and consequentially relational goods*_n and service*_n data;
second, said interactive data-rendering and data-editing functions attaching to and associating with said SIVSF and SIUNDV wherein said interactive data-rendering and data-editing functions successively correlating said individual compilations a relations of subset*_n with comprehensive compilations of relations of subset*_n; and
individual groups of said compilations of relations of subset*_n with comprehensive compilations of relations of subset*_n by said interactive data-rendering and data-editing functions add, drag and drop, scroll, undo, cut, copy, paste, delete, find, find next search, replace, go to, select data in said subset*-N of chronologically ordered relational sequences of timestamped data including images and videos and related goods*_n and service*_n data expressed and displayed in said interactive framework lines, nodes aka data points, spectrums, symbols, geometric configurations, tree traversals and graphs in and among said SIVSF and SIUNDV.

16. In the system according to claim 1, a method of networked repositories diagnosing, surveil ling, monitoring, trending patterning, preventing, predicting, extrapolating, controlling and doctoring acute, subacute and chronic diseases, disorders, inflammations, traumas, sequelae and related predilections, trends, patterns, options, predictions and extrapolations of said renderings and related procurements, prescriptions, utilizations, compliances and efficacies of related goods and services in people's lives henceforth collectively known as Abiotic Intelligent-Rendered Oracle also known as AIR Oracle, said networked repositories successively rendering adaptable, changeable, expandable and retractable dynamic multi-dimensional interactive universal network data-driven visualizations comprising successive interactive universal network data-driven flowingframeworks of visual-hierarchied flowingdata of connected visual-hierarchied data points aka visual-hierarchied nodes collectively known as SIUNDV in and among connected constellations wherein infinite individual connected assortments of interactive adaptable, changeable, expandable and retractable multi-dimensional dynamic network data-driven visualizations of visual-hierarchied flowingframeworks of visual-hierarchied flowingdata of connected interactive visual-hierarchied nodes having interactive dynamic adaptable and changeable projecting flowingframeworks building blocks henceforth collectively known as successive integral visual-hierarchied silhouetted flowingframeworks aka SIVSF integral in and to said SIUNDV wherein said SIVSF diagnosing, surveilling, monitoring, trending, patterning, preventing, predicting, extrapolating, controlling and doctoring, said AIR Oracle according successive outcomes being successively rendered by processor units correlating and relating individual compilations of relations of subset*_n of data comprising successive correlations of relations of successively rendered, correlated and related subset*_n of data by said processor units henceforth compilations of relations, individual groups of said compilations of relations with comprehensive compilations of relations first in and among said networked repositories and, second, in and among said SIVSF silhouetting said SIUNDV in and among said touchscreens, viewing screens and memories, said SIVSF including said projecting flowingframeworks building blocks diagnosing, surveilling, monitoring, trending, patterning, preventing, predicting, extrapolating, controlling and doctoring said AIR Oracle in and along said SIUNDV based on said successive outcomes, said method comprising processes of:
networking said networked devices comprising networked servers, smartphones, laptops and desktops, having said networked repositories, said touchscreens and viewing screens, cameras, microphones, said networked repositories comprising said processor units rendering said networks based on said compilations of relations, said processor units processing, tendering and expanding said individual and individual groups of compilations of relations with said comprehensive compilations of relations, the innovative computer programs attaching to said processor units attached to said memories, said innovative computer programs attaching to said processor units, said innovative computer programs instructing said processor units to successively render and expand said compilations of relations in said connected constellations and in and among said memories, said memories storing said individual and individual groups of compilations of relations with said comprehensive compilations of relations in said successive SIVSF*_n and said IUDDV*_n where * and _n represent diverse and plurality, respectively, and memories, software including said innovative computer programs, commercial operating systems managing said networked servers and devices, the Internet, commercial networking means security means and software including web browsers:
said innovative computer programs instructing said processor units to successively and continuously:
rendering AIR Oracle App activating innovative computer programs said processor units executing all functions described herein;
rendering the thesaurus software, said thesaurus software networking and grouping said networked repositories according to correlations of of same, similar, linked, related and differential relational subset*_n of data in and among memories of said networked repositories, said thesaurus software collaborating with said networked repositories to successively rendering, predicting, surveilling, monitoring, trending, patterning, preventing, predicting, extrapolating, controlling, and doctoring said AIR Oracle and said compilations of relations based on said thesaurus software successively rendering said compilations of relations in professionals' and corresponding laypeople's languages in and among said SIVSF successively silhouetting SIUNDV and said SIUNDV;
rendering a plurality of interactive electronic templates for rendering and successively rendering said successive outcomes based on said correlations of individual compilations of relations and individual groups of said individual compilations of relations with comprehensive compilations of relations based on correlations of linked, related and differential medicine-health-healthcare relational subset*_n comprising same, similar, linked, related and distinguishing data on chronologically ordered relational sequences of timestamped data including images and videos and relational subset*_n of goods*_n and service*_n data inherently, phenotypically and consequentially related to said medicine-health-healthcare relational subset*_n rendered by said processor units and stored in and among said memories, where symbols <, * and _n represent related to, diverse and plurality, respectively;
rendering said interactive data-rendering and data-editing functions attaching to and associating with said SIVSF avid SIUNDV, said interactive data-rendering and data-editing functions for successively render, add, drag and drop, scroll undo, cut, copy, paste, delete, find, find next, search, replace, go to, select said chronologically ordered relational sequences of timestamped data including images and videos and related subset*_n of data in and among said interactive framework lines, nodes aka data points, spectrums, symbols, geometric configurations, tree traversals and graphs, said data-rendering and data-editing functions for successively rendering, adapting, editing and expanding said successive outcomes in and among said memories and, consequently, in and among said SIVSF and SIUNDV;
scanning, matching and relating same, similar and distinguishing chronologically ordered relational sequences of timestamped data in and among said relational subset*_n of data in said individual compilations of relations and said individual groups of compilations of relations with said relational subset*_n of data in comprehensive compilations of relations to successively render said successive outcomes of said successive scans, matches and relations, said successive outcomes successively rendering, adapting, adding and editing said SIVSF integral in and to said SIUNDV and said SIUNDV in and among said touchscreens and viewing screens and, consequently, said compilations of relations in and among said memories;
rendering said constituent SIVSF silhouetting comprising said individual compilations of relations and said comprehensive compilations of relations, respectively, according to said successive outcomes being rendered by said processor units;
rendering said constituent SIVSF silhouetting SIUNDV comprising said individual groups of said compilations of relations and and said comprehensive compilations of relations, respectively, according to said successive outcomes being rendered by said processor units;
rendering said interactive dynamic adaptable and changeable projecting flowingframeworks building blocks to projecting said SIVSF to successively diagnosing, surveilling, monitoring, trending, patterning, presenting, predicting, extrapolating, controlling and doctoring said AIR Oracle in said SIVSF integral in and to SIUNDV and successively and progressively projecting said SIVSF to silhouet said SIUNDV according to said successive outcomes rendered by and among said processor units;
rendering, adapting, changing and expanding said successive outcomes in and among said memories and, consequently, in SIVSF integral in and to said SIUNDV and SIVSF successively silhouetting said SIUNDV wherein said successive outcomes are being continuously and perpetually rendered, adapted, changed and expanded by functions causing said SIVSF to successively diagnosing, surveilling, monitoring, adapting, trending, patterning, preventing, protecting, predicting, controlling and doctoring said AIR Oracle by said interactive framework lines nodes aka data, points, spectrums, symbols, geometric configurations, tree traversals and graphs interactively comprising, expressing and displaying chronologically ordered relational sequences of timestamped data including images and videos and related subset*_n of data of inherently, phenotypically and consequentially relational goods*_n and service*_n data specifically relevant to interactive framework lines, nodes aka data points, spectrums, symbols, geometric configurations, tree traversals and graphs wherein data are rendered interactive by said processor units; and
attaching and associating said interactive data-rendering and data-editing functions with said interactive framework lines, nodes aka data points, spectrums, symbols, geometric configurations, tree traversals and graphs wherein said interactive data-rendering and data-editing functions successively rendering, surveilling, monitoring, adapting, trending, patterning, predicting, extrapolating, controlling and doctoring said AIR Oracle by successively render, add, drag and drop, scroll, undo, cut copy, paste, delete, find, find next, search, replace, go to, select said chronologically ordered relational sequences of timestamped data including image, and videos and related subset*_n of data among said interactive framework lines, nodes aka data points, spectrums, symbols, geometric configurations tree traversals and graphs;
assigning said data-rendering and data-editing functions to cybersecurely rendering, expanding and editing said compilations of relations in and among said SIVSF integral in and to said SIUNDV and said memories by said data-rendering and data-editing functions defining cybersecured definitions, characteristics and limitations of data being rendered and permitted into said memories to prevent malfeasances and protect said system; and
rendering visual hierarchies in said SIVSF integral in and to said SIUNDV and SIUNDV to successively rendering, predicting, surveilling, monitoring, trending, patterning, preventing, predicting, extrapolating, controlling and doctoring said AIR Oracle and said compilations of relations in and among said memories.
